(19) United States
(12) Patent Application Publication
(10) Pub. No.: US 2023/0383327 A1
(43) Pub. Date: Nov. 30, 2023

(54) Method for Semisynthesis of NMN Involving Adenosine

(71) Applicant: RUBYBERRIES BIOTECHNOLOGIES CO., LTD, Zhongshan City, Guangdong (CN)

(72) Inventors: Caike JIN, Zhongshan City, Guangdong (CN); Yuan ZHAO, Zhongshan City, Guangdong (CN)

(21) Appl. No.: 18/031,610

(22) PCT Filed: Jun. 21, 2022
(86) PCT No.: PCT/CN2022/100140

§ 371 Date: Apr. 12, 2023

(30) Foreign Application Priority Data
Jun. 29, 2021 (CN) 202110728702.1

(51) Int. Cl. C12P 19/30

(52) U.S. Cl. CPC C12P 19/30

(57) ABSTRACT

A method for semisynthesis of NMN involving adenosine includes the steps in the same reaction system: (A) a step of adenosine, a phosphate and a sugar that can be metabolized by yeast cells reacting, catalyzed by yeast cells, to generate ATP; (B) a step of enzymatic phosphorylation of NR, and a corresponding step of NR reacting with ATP under the catalysis of NRK to generate NMN and ADP. In this way, efficient synthesis of NMN can be realized during a process of ATP generation and recycling, which can simplify the process and reduce emissions.

### CROSS REFERENCE OF RELATED APPLICATION

This is a U.S. National Stage under 35 U. S. C. 371 of the international Application Number PCT/CN2022/100140, filed Jun. 21, 2022, which claims priority under 35 U. S. C. 119(a-d) to Chinese application numbers 202110728702.1, filed Jun. 29, 2021, which are incorporated herewith by references in their entities.

### BACKGROUND OF THE PRESENT INVENTION

### Field of Invention

The present invention relates to the technical field of the synthesis of beta-nicotinamide mononucleotide (NMN), and more particularly to a method for semisynthesis of NMN involving adenosine.

### Description of Related Arts

β-nicotinamide mononucleotide (NMN) is the direct precursor for a human body to synthesize nicotinamide adenine dinucleotide (NAD). Supplementing NMN is the most effective way to increase the NAD level in the human body, it is beneficial for the metabolism of the human body and has broad and far-reaching health implications. Since the NAD levels in the elderly people are relatively low and sufficient NAD cannot be obtained from food, NMN is expected to become a dietary supplement for large-scale applications.

Currently, the conventional synthesis techniques of NMN include four methods: fermentation method, chemical synthesis method, semi-synthesis method and fully enzymatic method. Among them, the fermentation method needs to construct and produce NMN microbial strains, and in the process of mass culture and reproduction of the microorganisms, NMN is synthesized by bacterial cells. Because the basal activity of the key enzyme (NAMPT, nicotinamide phosphoribosyltransferase) that catalyzes the synthesis of NMN in various species, including low-level unicellular organisms, is generally very low, it is extremely difficult to construct a bacterial strain that highly expresses NMN. And because of the synthetic route is long and involves a multi-enzyme system and a natural decomposing enzyme system, so it is very difficult to produce NMN by efficient large-scale fermentation, and the process cost is high, and the product has no market competitiveness. The chemical synthesis method employs basic raw materials such as nicotinamide (or nicotinic acid), tetraacetyl ribose, and triphenoxyphos to first synthesize nicotinamide ribose (NR) by chemical methods, and then further phosphorylate NR to obtain NMN. The main problem of this method is that the chemical phosphorylation step of the second step involves inflammable, explosive and highly toxic substances, so that large-scale industrialization faces serious environmental protection and safety supervision problems, and there are also chemical enantiomer impurities, toxic residues of raw materials and solvents etc. The long-term safety concerns of human body application of its products are problems that are difficult to eliminate for consumers. The semi-synthetic method is to phosphorylate NR by enzymatic method on the basis of chemical synthesis of NR to obtain NMN. This method has the advantages and disadvantages of both chemical and enzymatic methods. The main problem is the residual risk of solvents and toxic components in the conventional chemical method, and the enzymatic phosphorylation step also requires expensive adenosine triphosphate (ATP), which is expensive. The fully enzymatic method uses nicotinamide, ribose and ATP as the basic raw materials, and uses a series of enzymes to catalyze the formation of NMN. The advantages of this method are environmental protection and safety, but the difficulty is that it involves the expression, purification and immobilization of various enzymes, and the cost of enzymes is high, another big problem with the fully enzyme method is that the amount of ATP is too large, which leads to the high cost of this method can becomes the main factor that hinder the fully enzymatic method to be promoted and used.

Among the four conventional synthetic methods of NMN, the semi-synthetic method is currently the mainstream method for synthesizing NMN. The starting materials of this method can be nicotinamide (or nicotinic acid) and tetraacetyl ribose, which are first synthesized into NR by chemical methods, and then NR and ATP are used to generate NMN under the catalysis of specific kinases, or NR is directly used as raw material for the production of NMN under the catalysis of a specific enzyme. The core step of the semi-synthetic method is the enzymatic phosphorylation of NR. ATP provides a phosphate group to NR to form NMN, and ATP becomes adenosine diphosphate (ADP). The reaction formula is: NR+ATP→*NMN+ADP, The enzyme that catalyzes this reaction is nicotinamide ribokinase (NRK). In order to reduce the amount of ATP, ADP and polyphosphate (sodium pyrophosphate, sodium tripolyphosphate or six tablets of sodium phosphate, etc.) are usually converted into ATP by enzymatic reaction to realize the reuse of ATP. The reaction formula is: ADP+PPi (pyrophosphate)→>ATP+Pi (phosphate), the enzyme that catalyzes this reaction is adenylate phosphotransferase (PPK2). These two steps of enzymatic reaction (phosphorylation of NR and regeneration of ATP) can be done separately or together. There are two main difficulties in the process. One is that the accumulation of a large amount of phosphate in the reaction will interfere with further reactions. The separation and removal of phosphate is difficult and affects the recovery rate of ATP. The other is that the reaction system involves two enzymes, which require a large amount of enzymes, and at the same time inevitably bring many mixed unwanted enzymes, and the degree of decomposition side reactions of NR, NMN, ATP, ADP, etc. is also high, and there will be nicotinamide, ribose, ADP, AMP, NR, adenosine, adenine and phosphate, etc. produced by side reactions in the reaction system, so that the system components become complex and difficult to control, resulting in difficulties in the purification process of NMN products and high costs, and the stability of product quality is also difficult to control.

### SUMMARY OF THE PRESENT INVENTION

The invention is advantageous in that it provides provide a method for semisynthesis of NMN involving adenosine, wherein compared with the conventional semesynthesis method, the method for semisynthesis of NMN involving adenosine can simplify the purification process of NMN products, so that it has a relatively low production cost.

Another advantage of the present invention is to provide a method for semisynthesis of NMN involving adenosine, wherein the method for semisynthesis of NMN involving adenosine takes into account the advantage of chemical method and enzymatic method to reduce discharge while guarantee the synthetic efficiency of NMN product, and correspondingly result in lower production cost and environmental cost.

Another advantage of the present invention is to provide a method for semisynthesis of NMN involving adenosine, wherein compared with the conventional semesynthesis method, the method for semisynthesis of NMN involving adenosine adopts cheap adenosine instead of ATP, and the way of introducing yeast cells to convert adenosine into ATP according to energy metabolism in the reaction can be combined with the conventional NR phosphorylation process to realize the reuse of ATP without the recovery process of ATP, and the phosphoric salt formed by the conventional NR phosphorylation process is used as a reactant, so as to eliminate the phosphate removal process, thus simplifying the purification process of NMN products based on the participation of the adenosine.

Another advantage of the present invention is to provide a method for semisynthesis of NMN involving adenosine, wherein compared with the conventional semesynthesis method, the method for semisynthesis of NMN involving adenosine adopts cheap adenosine instead of ATP, and the way of introducing yeast cells to convert adenosine into ATP according to energy metabolism in the reaction can be combined with the conventional NR phosphorylation process to realize the reuse of ATP and reduce the consumption of adenosine, and because the price of the adenosine is much lower than that of ATP, the raw material cost of the corresponding NMN product is significantly reduced, so that it has a relatively low production cost.

Another advantage of the present invention is to provide a method for semisynthesis of NMN involving adenosine, wherein compared with the conventional semesynthesis method, the method for semisynthesis of NMN involving adenosine adopts cheap adenosine instead of ATP, and the way of introducing yeast cells to convert adenosine into ATP according to energy metabolism in the reaction can be combined with the conventional NR phosphorylation process to realize the reuse of ATP and the phosphate formed by the conventional NR phosphorylation process can be used as a reactant, after separating and purifying NMN, the remaining reactants and resultants can be reused to reduce unwanted emissions, and the production of corresponding NMN products is environmentally friendly and has low environmental costs.

Another advantage of the present invention is to provide a method for semisynthesis of NMN involving adenosine, wherein the method for semisynthesis of NMN involving adenosine of the present invention adopts NR, phosphate, adenosine and sucrose as raw material, and takes NRK and yeast cells as catalysts, the generation of ATP, NR phosphorylation and the utilization of ATP are carried out in one reaction system, and the efficient synthesis of NMN can be completed. While taking into account the advantages of chemical methods to ensure the synthesis efficiency of NMN products, various reactants (NR, phosphate, adenosine, sucrose, etc.) can be substantially completely consumed to take into account the advantages of the enzymatic method to reduce unwanted emissions, so that it is simpler and cheaper than the conventional semi-synthetic method, it is simple and easy to implement, and the cost is low.

According to an aspect of the present invention, the present invention provides a method for semisynthesis of NMN involving adenosine which comprises the following steps in a same reaction system:

(A) generating ATP by the reaction of adenosine, phosphate and carbohydrate which is capable of being metabolized by yeast cells under the catalysis of the yeast cells; and

(B) carrying out an enzymatic phosphorylation step of NR in which NR and ATP react to produce NMN and ADP under the catalysis of NRK.

In one embodiment, in the reaction system of the method for semisynthesis of NMN involving adenosine, the NR raw material is selected from at least one of commercial NR pure products, NR-containing solids, and NR-containing liquids.

In one embodiment, in the reaction system of the method for semisynthesis of NMN involving adenosine, the carbohydrate metabolized by the yeast cells is selected from at least one of glucose, sucrose, starch and glycerol.

In one embodiment, in the reaction system of the method for semisynthesis of NMN involving adenosine, the NRK enzyme exists in at least one original form of liquid enzyme form and immobilized enzyme form.

In one embodiment, wherein in the reaction system of the method for semisynthesis of NMN involving adenosine, the yeast cells are yeast cells capable of oxidative phosphorylation metabolism.

In one embodiment, in the reaction system of the method for semisynthesis of NMN

involving adenosine, the yeast cells are selected from at least one of Pichia pastoris and Saccharomyces cerevisiae.

In one embodiment, metal ion is further added to the reaction system of the method for semisynthesis of NMN involving adenosine.

In one embodiment, in the reaction system of the method for semisynthesis of NMN involving adenosine, the added metal ion is at least one selected from magnesium ion and manganese ion.

In one embodiment, in the reaction system of the method for semisynthesis of NMN involving adenosine, the molar ratio of adenosine to NR ranges from 0.01 to 1.

In one embodiment, in the reaction system of the method for semisynthesis of NMN involving adenosine, the molar ratio of NR to phosphate ranges from 1 to 20.

In one embodiment, in the reaction system of the method for semisynthesis of NMN involving adenosine, the yeast cells are wet yeasts once being stored cryogenically.

In one embodiment, at least one organic reagent of toluene, n-butanol and Tween 20 is further added to the reaction system of the NMN semi-synthesis method involving adenosine.

In one embodiment, wherein the step (A) is initiated before the step (B) to provide ATP for the reaction of the step (B), so as to form a state in which the step (A) and the step (B) are in the same reaction system to be beneficial to promote each other.

In one embodiment, the method for semisynthesis of NMN involving adenosine further comprises a step of regenerating ADP and phosphate into ATP under the action of the yeast cells.

### DETAILED DESCRIPTION OF THE PREFERRED EMBODIMENT

The following description is disclosed to enable any person skilled in the art to make and use the present invention. Preferred embodiments are provided in the following description only as examples and modifications will be apparent to those skilled in the art. The general principles defined in the following description would be applied to other embodiments, alternatives, modifications, equivalents, and applications without departing from the spirit and scope of the present invention.

Those skilled in the art should understand that, in the disclosure of the present invention, terminologies of “longitudinal,” “lateral,” “upper,” “front,” “back,” “left,” “right,” “perpendicular,” “horizontal,” “top,” “bottom,” “inner,” “outer,” and etc. that indicate relations of directions or positions are based on the relations of directions or positions shown in the appended drawings, which are only to facilitate descriptions of the present invention and to simplify the descriptions, rather than to indicate or imply that the referred device or element is limited to the specific direction or to be operated or configured in the specific direction. Therefore, the above-mentioned terminologies shall not be interpreted as confine to the present invention.

It is understandable that the term “a” or “an” should be understood as “at least one” or “one or more”. In other words, in some embodiments, the number of an element can be one and in other embodiment the number of the element can be more than one. The term “a” or “an” is not construed as a limitation of quantity.

The present invention provides a method for semisynthesis of NMN involving adenosine, compared with the conventional semisynthesis method, the method for semisynthesis of NMN involving adenosine of the present invention adopts cheap adenosine instead of ATP, and yeast cells are introduced in the reaction to convert adenosine into ATP according to energy metabolism, so that it can combine the conventional NR phosphorylation process to realize the reuse of ATP and utilize the phosphate formed by the conventional NR phosphorylation process as a reactant.

Specifically, the method for semisynthesis of NMN involving adenosine adopts NR, phosphate, adenosine, and carbohydrate (such as glucose, sucrose, and glycerol, etc.) that can be metabolized by yeast cells as raw materials, and NRK and the yeast cells as Catalysts, the generation of ATP, NR phosphorylation and the utilization of ATP are unified in one reaction system, and the efficient synthesis of NMN can be completed. The reaction formula is: NR+sucrose+adenosine+phosphate+O2→NMN+ATP+CO2+H2O. In this reaction system, the yeast cells use carbohydrate oxidation to provide energy through oxidative phosphorylation to actuate the combination of phosphate and adenosine to generate adenosine monophosphate (AMP), and then generate ADP and ATP, and ATP participates in the phosphorylation of NR. After becoming ADP, it is automatically converted into ATP to continue to participate in the reaction. In other words, adenosine, AMP, and ADP in the reaction system can be quickly converted into ATP that can participate in the phosphorylation of NR. Compared with the conventional semisynthesis method, the phosphate formed during the phosphorylation of NR can be used as a reactant, so that the removal process of phosphate is omitted, and the reuse of ATP can be realized without the recovery process of ATP, so the purification process of the NMN product is simplified based on the participation of the adenosine.

Furthermore, in one embodiment of the present invention, the method for semisynthesis of NMN involving adenosine adopts NR, phosphate, adenosine, sucrose and magnesium ions as raw materials, and NRK (not limited to liquid enzyme or immobilized enzyme)) and the yeast cells as catalysts, the initial pH of the aqueous solution is in the neutral range, and the reaction is carried out in contact with air and stirring. Then the generation of ATP, NR phosphorylation and the utilization of ATP are carried out in one reaction system, and various reactants (NR, phosphate, adenosine, sucrose, etc.) can be substantially completely consumed, and the corresponding reaction system is simple and easy to operate, and the cost is low, and is environmentally friendly and has a low environmental cost.

In another embodiment of the present invention, the method for semisynthesis of NMN involving adenosine adopts NR and adenosine as substrates, and uses yeast and nicotinamide to ribokinase to produce NMN in a one-pot method. Illustratively, NRC with a final concentration of 100 mM, 50 mM adenosine, 330 mM dipotassium hydrogen phosphate, 70 mM potassium dihydrogen phosphate, 120 mM sucrose, 50 mM magnesium chloride, 5 mM manganese chloride, 300 g yeast, 500 mg nicotinamide ribokinase crude enzyme freeze-dried powder are sequentially added to the 1 L reaction system, after fully stirring and dissolving, control the reaction temperature to 37° C., 300 rpm stirring reaction, use a high performance liquid chromatography to detect the concentration of NMN during the reaction, the reaction ends within six hours, and the reaction yields 29.84 g of NMN. The yield rate is 89.3%.

In another embodiment of the present invention, the method for semisynthesis of NMN involving adenosine adopts NR and adenosine as substrates, and uses Saccharomyces cerevisiae and nicotinamide ribokinase magnetically immobilized enzyme to produce NMN in a one-pot method. Illustratively, add adenosine with a final concentration of 50 mM, 330 mM potassium dihydrogen phosphate, 70 mM potassium dihydrogen phosphate, 120 mM sucrose, magnesium chloride, 5 mM manganese chloride, and 300 g wet Saccharomyces cerevisiae in sequence in a 1 L reaction system. After fully stirring and dissolving, control the reaction temperature to 37° C., and let it stand for fermentation for one hour. Add NRC with a final concentration of 100 mM and 300 g of nicotinamide ribokinase magnetically immobilized enzyme to the above yeast fermentation broth, stir the reaction at 300 rpm, control the reaction temperature at 37° C., and use an automatic titrator to control the reaction pH to be 6.0 with 3M sodium hydroxide. During the reaction process, the NMN concentration is detected by the high performance liquid chromatography, and the reaction is completed within two hours, and 31.58 g of NMN is obtained from the reaction, and the reaction conversion rate is 94.5%.

To further describe the present invention, the method for semisynthesis of NMN involving adenosine comprises the following steps under the same reaction system:

(A) generating ATP by adenosine, phosphate and carbohydrate which is capable of being metabolized by yeast cells under the catalysis of the yeast cells; and

(B) carrying out an enzymatic phosphorylation step of NR in which NR and ATP react to produce NMN and ADP under the catalysis of NRK.

It can be understood that in the reaction system of the method for semisynthesis of NMN involving adenosine, the NR raw material is selected from at least one of commercial NR pure products, NR-containing solids, and NR-containing liquids.

Furthermore in the reaction system of the method for semisynthesis of NMN involving adenosine, the carbohydrate metabolized by the yeast cells is selected from at least one of glucose, sucrose, starch, glycerol and the combination thereof.

Particularly, in the reaction system of the method for semisynthesis of NMN involving adenosine, the NRK enzyme exists in at least one original form of liquid enzyme form and immobilized enzyme form, the present invention is not limited in this aspect.

Furthermore, in the reaction system of the method for semisynthesis of NMN involving adenosine, the yeast cells are yeast cells capable of oxidative phosphorylation metabolism, such as Pichia pastoris and Saccharomyces cerevisiae.

Alternatively, metal ion is further added to the reaction system of the method for semisynthesis of NMN involving adenosine, such as magnesium ion and manganese ion.

Preferably, in the reaction system of the method for semisynthesis of NMN involving adenosine, the molar ratio of adenosine to NR ranges from 0.01 to 1.

Preferably, in the reaction system of the method for semisynthesis of NMN involving adenosine, the molar ratio of NR to phosphate ranges from 1 to 20.

It is worth mentioning that in the reaction system of the method for semisynthesis of NMN involving adenosine, the yeast cells can be wet yeasts once being stored cryogenically.

Particularly, at least one organic reagent of toluene, n-butanol and Tween 20 is further added to the reaction system of the NMN semi-synthesis method involving adenosine.

It is worth mentioning that in some embodiments, in the reaction procedure, the step (A) is initiated before the step (B) to provide ATP for the reaction of the step (B), so as to form a state in which the step (A) and the step (B) are in the same reaction system to be beneficial to promote each other.

Particularly, according to some embodiments of the present inventions, the method for semisynthesis of NMN involving adenosine further comprises a step of regenerating ADP and phosphate into ATP under the action of the yeast cells.

One skilled in the art will understand that the embodiment of the present invention as shown in the drawings and described above is exemplary only and not intended to be limiting.

It will thus be seen that the objects of the present invention have been fully and effectively accomplished. The embodiments have been shown and described for the purposes of illustrating the functional and structural principles of the present invention and are subject to change without departure from such principles. Therefore, this invention includes all modifications encompassed within the spirit and scope of the following claims.

1. A method for semisynthesis of NMN involving adenosine which comprises the following steps in a same reaction system:
(A) generating ATP by the reaction of adenosine, phosphate and carbohydrate which is capable of being metabolized by yeast cells under the catalysis of the yeast cells; and
(B) carrying out an enzymatic phosphorylation step of NR in which NR and ATP react to produce NMN and ADP under the catalysis of NRK.

2. The method for semisynthesis of NMN involving adenosine according to claim 1, wherein in the reaction system of the method for semisynthesis of NMN involving adenosine, a raw material of NR is selected from at least one of commercial NR pure products, NR-containing solids, and NR-containing liquids.

3. The method for semisynthesis of NMN involving adenosine according to claim 1, wherein in the reaction system of the method for semisynthesis of NMN involving adenosine, the carbohydrate metabolized by the yeast cells is selected from at least one of glucose, sucrose, starch and glycerol.

4. The method for semisynthesis of NMN involving adenosine according to claim 1, wherein in the reaction system of the method for semisynthesis of NMN involving adenosine, the NRK enzyme exists in at least one original form of liquid enzyme form and immobilized enzyme form.

5. The method for semisynthesis of NMN involving adenosine according to claim 1, wherein in the reaction system of the method for semisynthesis of NMN involving adenosine, the yeast cells are yeast cells capable of oxidative phosphorylation metabolism.

6. The method for semisynthesis of NMN involving adenosine according to claim 5, wherein in the reaction system of the method for semisynthesis of NMN involving adenosine, the yeast cells are selected from at least one of Pichia pastoris and Saccharomyces cerevisiae.

7. The method for semisynthesis of NMN involving adenosine according to claim 1, wherein metal ion is further added to the reaction system of the method for semisynthesis of NMN involving adenosine.

8. The method for semisynthesis of NMN involving adenosine according to claim 7, wherein in the reaction system of the method for semisynthesis of NMN involving adenosine, the added metal ion is at least one selected from magnesium ion and manganese ion.

9. The method for semisynthesis of NMN involving adenosine according to claim 1, wherein in the reaction system of the method for semisynthesis of NMN involving adenosine, the molar ratio of adenosine to NR ranges from 0.01 to 1.

10. The method for semisynthesis of NMN involving adenosine according to claim 9, wherein in the reaction system of the method for semisynthesis of NMN involving adenosine, the molar ratio of NR to phosphate ranges from 1 to 20.

11. The method for semisynthesis of NMN involving adenosine according to claim 1, wherein in the reaction system of the method for semisynthesis of NMN involving adenosine, the yeast cells are wet yeasts once being stored cryogenically.

12. The method for semisynthesis of NMN involving adenosine according to claim 1, wherein at least one organic reagent of toluene, n-butanol and Tween 20 is further added to the reaction system of the NMN semi-synthesis method involving adenosine.

13. The method for semisynthesis of NMN involving adenosine according to claim 1, wherein the step (A) is initiated before the step (B) to provide ATP for the reaction of the step (B), so as to form a state in which the step (A) and the step (B) are in the same reaction system to be beneficial to promote each other.

14. The method for semisynthesis of NMN involving adenosine according to claim 1, wherein the method for semisynthesis of NMN involving adenosine further comprises a step of regenerating ADP and phosphate into ATP under the action of the yeast cells.

15. The method for semisynthesis of NMN involving adenosine according to claim 2, wherein the method for semisynthesis of NMN involving adenosine further comprises a step of regenerating ADP and phosphate into ATP under the action of the yeast cells.

16. The method for semisynthesis of NMN involving adenosine according to claim 3, wherein the method for semisynthesis of NMN involving adenosine further comprises a step of regenerating ADP and phosphate into ATP under the action of the yeast cells.

17. The method for semisynthesis of NMN involving adenosine according to claim 4, wherein the method for semisynthesis of NMN involving adenosine further comprises a step of regenerating ADP and phosphate into ATP under the action of the yeast cells.

18. The method for semisynthesis of NMN involving adenosine according to claim 5, wherein the method for semisynthesis of NMN involving adenosine further comprises a step of regenerating ADP and phosphate into ATP under the action of the yeast cells.

19. The method for semisynthesis of NMN involving adenosine according to claim 6, wherein the method for semisynthesis of NMN involving adenosine further comprises a step of regenerating ADP and phosphate into ATP under the action of the yeast cells.

20. The method for semisynthesis of NMN involving adenosine according to claim 7, wherein the method for semisynthesis of NMN involving adenosine further comprises a step of regenerating ADP and phosphate into ATP under the action of the yeast cells.
